(19) United States
(12) Patent Application Publication
(10) Pub. No.: US 2007/0231381 A1
(43) Pub. Date: Oct. 4, 2007

(54) Controlled-release dosage forms comprising zolpidem or a salt thereof

(76) Inventor: Bernard Charles Sherman, Toronto (CA)

Correspondence Address: NIXON & VANDERHYE, PC, 901 NORTH GLEBE ROAD, 11TH FLOOR, ARLINGTON, VA, 22203, US

(21) Appl. No.: 11/393,731

(22) Filed: Mar. 31, 2006

(51) Int. Cl. A61K 31/4745; A61K 9/20

(52) U.S. Cl. 424/464; 514/303

(57) ABSTRACT

Controlled-release zolpidem compositions comprising granules that comprise zolpidem or a salt thereof, a water-insoluble polymer, and an enteric polymer.

### BACKGROUND OF THE INVENTION

Zolpidem is a hypnotic drug that may be administered orally.

It has been sold in the United States and elsewhere in immediate-release tablets containing 5 mg and 10 mg of zolpidem tartrate under the tradename Ambien™.

More recently, controlled release tablets have been introduced into the U.S. market under the tradename Ambien CR™. These tablets contain zolpidem tartrate in strengths of 6.25 mg and 12.5 mg.

These tablets are two-layer tablets, and are made following the teaching of U.S. Pat. No. 6,514,531. One layer contains approximately 70% of the zolpidem tartrate content and releases the content immediately after ingestion; the other layer allows for slower release of the balance of the drug content. The purpose of the slower-release layer is to provide extended plasma concentrations beyond three hours after administration.

The manufacture of two-layer tablets requires use of specialized equipment not available in most pharmaceutical factories.

The purpose of the present invention is thus to enable a simpler method of manufacture of tablets comprising zolpidem or a salt thereof that, after ingestion, will provide prompt-release of most of the drug content and slower release of the balance of the drug content.

### DESCRIPTION OF THE INVENTION

Compositions of the present invention are tablets that comprise granules, wherein the granules are comprised of zolpidem or a salt thereof, a water-insoluble polymer, and an enteric polymer.

An enteric polymer will be understood to mean a polymer that is insoluble in aqueous media at pH below about 4, but soluble at basic pH of above 6. Examples of such polymers are polyvinyl acetate phthalate and cellulose acetate phthalate. Most preferred is polyvinyl acetate phthalate.

Suitable water-insoluble polymers will include, for example, ethylcellulose and cellulose acetate. Most preferred is ethylcellulose.

The granules comprising zolpidem or a salt thereof, a water-soluble polymer, and an enteric polymer will preferably be made by compaction and milling. That is to say, the ingredients will be mixed together and compacted, and the compacted material will then be milled into granules of a desired size.

In addition to the granules, the tablets will comprise a disintegrant such as, for example, croscarmellose sodium, sodium starch glycolate, and crospovidone. Most preferred is crospovidone. The purpose of the disintegrant is to cause the tablets to disintegrate to release the granules in gastrointestinal fluid after ingestion.

The total amount of the two polymers by weight in the granules will preferably substantially exceed the amount of the zolpidem or sale thereof.

The ratio of enteric polymer to water insoluble polymer by weight will preferably be between 1:9 and 9:1 and will more preferably be between 2:8 and 8:2, will even more preferably be between 4:6 and 6:4, and will most preferably be about 1:1.

Gastric fluid is acidic. After ingestion, a tablet will disintegrate in gastric fluid to release the granules. However, because the granules are comprised largely of the water-insoluble polymer and the enteric polymer, the granules will not dissolve or disintegrate in the gastric fluid. Nevertheless, the content of zolpidem or a salt thereof that is at or near the surface of the granules will rapidly leach out of the granules and dissolve in the gastric fluid. The remaining amount of zolpidem or a salt thereof that is deeper within the granules will not rapidly leach out, but will remain within the granules in the gastric fluid. The fraction of the content of the zolpidem or salt thereof that is rapidly released in this manner can be controlled by controlling the size of the granules. i.e. the larger the granules are, the less will be the fraction of the drug content that will rapidly dissolve in this manner, and the smaller the granules are, the higher the fraction that will rapidly dissolve in this manner.

As the granules progress through the gastrointestinal tract, they will reach the small intestine, where pH will be above 6. At this pH, the enteric polymer in the granules is soluble. Hence, the enteric polymer will begin to leach out of the granules, and along with it will come more of the zolpidem or salt thereof. Hence, when the granules reach the small intestine, the balance of the drug that did not dissolve in the stomach will gradually leach out and dissolve. The rate of dissolution in the intestine can be controlled by varying the ratio of enteric polymer to water-insoluble polymer. A higher ratio will give more rapid dissolution in the intestinal fluid, and a lower ratio will give slower dissolution.

It can thus be seen that the present invention enables biphasic release, which enables tablets that are therapeutically equivalent to Ambien CR™ tablets, but use a different mechanism to control release.

The invention will be better understood from the following examples, which are intended to be illustrative, and not limiting.

### EXAMPLE 1

Zolpidem tartrate, ethylcellulose and polyvinylacetate phthalate were mixed in the proportions as follows:

| | |
|---|---|
| Zolpidem tartrate | 10.0 |
| Ethylcellulose | 45.0 |
| Polyvinyl acetate phthalate | 45.0 |
| | 100.0 |

The mixture was compacted, milled, and sifted to select the granules that pass through mesh #20 screen (20 wires per mesh) but do not pass through a #30 screen.

### EXAMPLE 2

The granules from example 1 were mixed with crospovidone and magnesium stearate in the proportions as follows:

| | |
|---|---|
| Granules | 125.0 |
| Crospovidone | 34.2 |
| Magnesium stearate | 0.8 |
| | 160.0 |

The mixture was compressed into tablets of weight 125 mg, so that each tablet contained 12.5 mg of zolpidem tablets.

These tablets were tested for dissolution to 12 hours in a type 2 dissolution apparatus according to the U.S. Pharmacopoeia at 50 rpm in both 0.1N HCl and phosphate buffer pH 6.8.

In 0.1 N HCl, the amount dissolved was about 60% in 1 h, and then increased only a few percent over the balance of 12 hrs. In phosphate buffer pH 6.8, dissolution was also about 60% in 1 h, but the amount dissolved continued to gradually increase to about 90% at 4 h and 95% at 8 h.

1. A controlled release composition comprising granules that comprise zolpidem or a salt thereof, a water-insoluble polymer, and an enteric polymer.

2. A composition of claim 1 in the form of a tablet.

3. A composition of claim 1 or 2 that further comprises a disintegrant.

4. A composition of any of claims 1 to 3 wherein the water-insoluble polymer is ethylcellulose.

5. A composition of any of claim 1 to 4 wherein the enteric polymer is polyvinyl acetate phthalate.

6. A composition of any of claims 1 to 5 wherein the ratio of enteric polymer to water-insoluble polymer by weight is from 1:9 to 9:1.

7. A composition of claim 6 wherein the ratio is from 2:8 to 8:2.

8. A composition of claim 7 wherein the ratio is from 4:6 to 6:4.

9. A composition of claim 8 wherein the ratio of about 1:1.

10. A composition of any of claims 1 to 9 wherein the disintegrant is crospovidone.

11. A composition of any of claims 1 to 9 wherein the disintegrant is croscarmellose sodium.
